(19) United States
(12) Patent Application Publication
(10) Pub. No.: US 2006/0100632 A1
(43) Pub. Date: May 11, 2006

(54) Apparatus and method for shaping a mammalian joint surface

(76) Inventor: Barry M. Fell, Hummelstown, PA (US)

Correspondence Address: BROOKS KUSHMAN P.C., 1000 TOWN CENTER, TWENTY-SECOND FLOOR, SOUTHFIELD, MI, 48075, US

(21) Appl. No.: 10/985,763

(22) Filed: Nov. 9, 2004

(51) Int. Cl. A61B 17/16

(52) U.S. Cl. 606/81

(57) ABSTRACT

An apparatus and method are provided for shaping a mammalian joint surface, such as within the knee joint. The apparatus includes a shaft having a proximal end and a distal end, and a head pivotally connected to the shaft distal end. The head includes a textured portion arranged to contact and shape the joint surface.

### BACKGROUND OF THE INVENTION

1. Field of the Invention

This invention relates to an apparatus and method for shaping a mammalian joint surface, such as prior to implantation of a prosthetic device.

2. Background Art

A joint generally includes two bony structures spanned by soft tissues that hold the bone structures together and aid in defining the motion of the joint. In the knee, soft tissues such as ligaments, tendons, and menisci provide support to the tibia and femur bones, where the tibia and femur are covered by articular and meniscal cartilage.

Articular cartilage and meniscal cartilage provide the mobile weight bearing surfaces of the knee joint. Damage to these surfaces can be caused by genetic predisposition, trauma, and/or aging. The result is usually thinning and softening of the articular cartilage, and degenerative tearing of the meniscal cartilage. In patients with osteoarthritis, the degenerative process typically leads to an asymmetric wear pattern that leaves one compartment with significantly less articular cartilage covering the weight bearing areas of the tibia and femur than the other compartment. Most commonly, the medial compartment of the knee joint is affected more than the lateral compartment.

Osteoarthritis is usually defined in stages of Grade I through V, with Grade III revealing significant articular cartilage loss, Grade IV revealing some eburnation of the subchondral bone, and Grade V detailing both significant articular loss and bone loss. The disease manifests itself as periodic to continuous pain that can be quite uncomfortable for the patient. Increasing joint laxity is suspected of causing some of the pain one feels. In addition, as the bearing loads are shifted, the body responds to the increased loading on the diseased compartment with an increased production of bony surface area (osteophytes) in an attempt to reduce the area unit loading. All of this shifting of the knee (joint) component geometry causes a misalignment of the mechanical axis of the joint. This misalignment causes an increase in the rate of degenerative change to the diseased joint surfaces, causing an ever-increasing amount of cartilage debris and osteophytes to build up in the joint, and further causing joint inflammation and subsequent pain.

Various methods of treatment are available to treat these disease processes. Treatment of the affected bone surfaces depends, among other things, upon the severity of the damage to the articular surface and the age and physical condition of the patient. Surgery wherein the articulating elements of the joint are replaced with artificial elements is often indicated. For example, U.S. Pat. Nos. 6,206,927 and 6,558,421 and copending U.S. application Ser. No. 10/232,608 disclose a prosthesis for the knee compartment which fills the joint space in order to replace the missing articular materials. This prosthesis provides an anatomically correct bearing surface for both the tibial plateau and femoral condyle to articulate against.

Prior to receiving an implantable prosthesis, or in preparation for other surgical procedures, various instruments have been developed for use in shaping the joint surfaces involved. In particular, shaping devices or rasps are utilized for smoothing or texturing a mammalian joint surface, including bone and/or cartilage. Smoothing a diseased or damaged bone surface to remove osteophytes can decrease the friction between the prosthesis and the bone surface. Osteophytes can cause damage by encroaching into the joint space or breaking loose and becoming tissue debris, and can result in accelerated degradation of the prosthetic surface.

In order to sufficiently shape a joint surface, current rasps and shaping devices require a relatively large incision in order to position the device at all necessary angles and are cumbersome to position appropriately. In addition, current shaping devices often require fixation to some joint surface for location and/or reference during the cutting and shaping procedure. What is needed is an apparatus and method for shaping a joint surface that allows for precise shaping of the surface while being easily maneuverable through a small incision and within the limited space available among the joint tissues, which does not require fixation, and which is cost effective and easy to use.

### SUMMARY OF THE INVENTION

Accordingly, an apparatus for shaping a mammalian joint surface, such as bone or cartilage, is provided which includes a shaft having a proximal end and a distal end, and a head pivotally connected to the shaft distal end. The head includes a textured portion arranged to contact and shape the joint surface.

According to one aspect of the present invention, the head is connected to the shaft distal end by a hinge joint. Alternatively, a universal joint or a ball joint can be utilized to pivotally connect the shaft to the head. The head can be generally square or rectangular, or alternatively can be round, hemispherical, spherical, hourglass, cylindrical, or ball-shaped. The head can include a medial- lateral radius of curvature and, additionally or alternatively, an anterior-posterior radius of curvature. The head can also include a compound curvature, could be generally concave, or could be generally flat.

The textured portion of the head can include a plurality of cutting members, such as ridges. The textured portion can be integrally formed on the head, or the textured portion can comprise a separate portion that is attached to the head. The cutting members can include openings for allowing tissue debris to exit away from the joint surface.

The shaft can be generally cylindrical, and can be arranged to be connected to a powered surgical instrument, such as a reciprocating, oscillating, rotating, or vibrating saw. The apparatus can be constructed from any material suitable for the intended purpose, such as a metallic material or a polymeric material. According to one aspect of the present invention, the apparatus is arranged for use in shaping at least one of the femoral surface and the tibial surface of the knee.

In accordance with the present invention, a rasp is provided for shaping a surface within a knee joint. The rasp includes an elongated shaft having a proximal end and a distal end, and a generally concave shaping head pivotally connected to the shaft distal end. The head includes cutting members for contacting and shaping the knee joint surface.

Correspondingly, a method of shaping a mammalian joint surface is provided. The method includes providing an apparatus including a shaft having a proximal end and a distal end, and a head pivotally attached to the shaft distal end, where the head has a textured portion. The method further includes placing the textured portion of the apparatus in contact with the joint surface for shaping the joint surface.

According to the present invention, placing the textured portion in contact with the joint surface can include pivoting the shaft relative to the head, and shaping the joint surface can include maneuvering the apparatus in a free-hand manner. In addition, shaping of the joint surface can be performed without requiring fixation of the apparatus to any joint surface. The method can also include connecting the shaft proximal end to a powered surgical instrument, and can include using a guide to aid in placement of the apparatus. Shaping of bone or cartilage, as well as shaping at least one of a femoral surface and a tibial surface of the knee, are contemplated. In addition, the method according to the present invention can include making an incision through which the apparatus is inserted into a joint cavity, and implanting a prosthesis following shaping of the joint surface.

The above features and advantages, along with other features and advantages of the present invention are readily apparent from the following detailed description of the invention when taken in connection with the accompanying drawings.

### BRIEF DESCRIPTION OF THE DRAWINGS

FIG. 1 is a top perspective view of an apparatus according to the present invention;

FIG. 2 is a bottom perspective view of the apparatus of FIG. 1;

FIG. 3 is a fragmentary side elevational view of a head of the apparatus in accordance with the present invention;

FIG. 4 is a top perspective view of an apparatus according to another aspect of the present invention; and

FIG. 5 is a top perspective view of an apparatus according to yet another aspect of the present invention; and

FIG. 6 is a side elevational view of the apparatus according to the present invention connected to a powered surgical instrument.

### DETAILED DESCRIPTION OF THE EMBODIMENT(S) OF THE PRESENT INVENTION

The present invention provides an apparatus and method for shaping a mammalian joint surface, which can include bone and/or cartilage. Advantageously, the apparatus and method of the present invention allow for free-hand shaping of a mammalian joint surface to a pre-determined final shape which is defined by the configuration of the shaping head, without the need to use jigs or fixtures or to affix the apparatus to another surface for location and/or reference.

The apparatus and method described herein can be utilized, for example, for preparing a mammalian joint surface for a surgical procedure, such as receiving an implantable prosthesis, wherein shaping can include smoothing, cutting, and/or texturing of the joint surface. According to one aspect of the present invention, the shaping apparatus can have dimensions and other properties suitable for use with one or more joint surfaces within the knee joint, and the apparatus is particularly arranged for use in shaping the femoral condylar surface. However, it is understood that the apparatus and method of the present invention can be utilized in joints other than the knee, such as the hip, shoulder, wrist, ankle, elbow, and others.

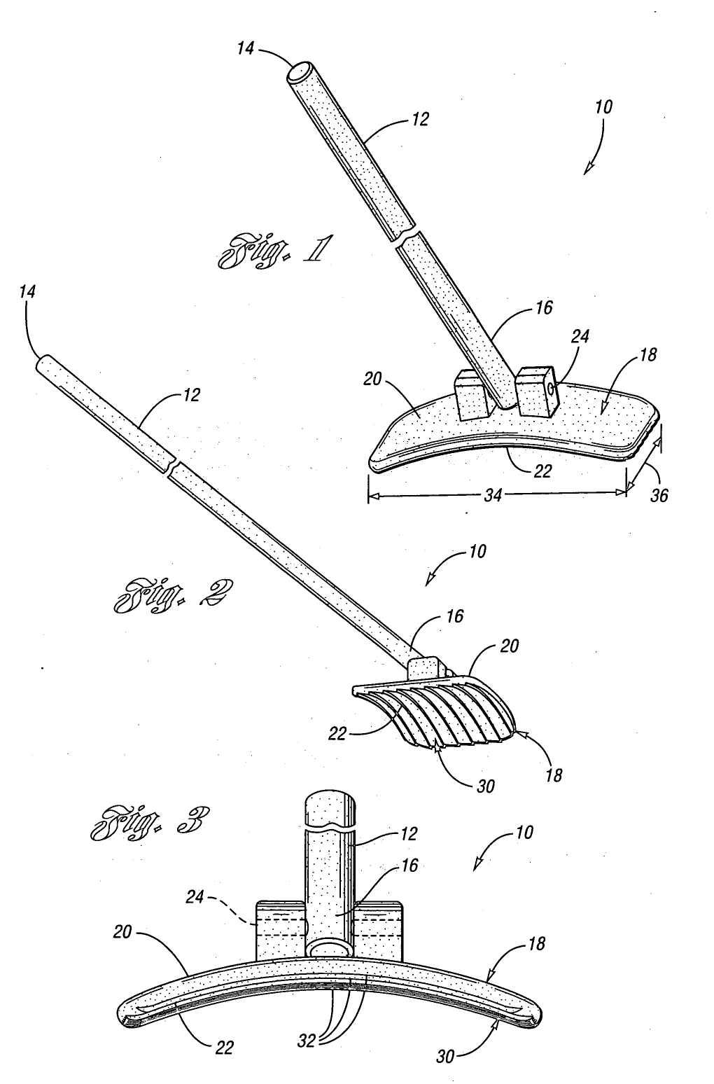

With reference first to FIGS. 1-3, perspective views of an apparatus 10 according to the present invention are depicted. As shown, apparatus 10 includes an elongated shaft 12 having a proximal end 14 and a distal end 16. Shaft distal end 16 is connected to a shaping head 18 where, according to one aspect of the present invention, head 18 can include a top surface 20 and a bottom surface 22. Advantageously, shaft 12 and head 18 are connected so as to allow pivotal movement therebetween to allow for easy maneuverability when shaping the joint surface.

In apparatus 10 depicted in FIGS. 1-3, shaft 12 and head 18 are connected via a hinge joint, with a pin 24 through shaft distal end 16 securing shaft 12 to head 18. This hinged connection allows shaft 12 to move pivotally with respect to head 18, thus allowing for shaping of the joint surface at angles not possible with prior art rasps which employ a shaft that is angularly fixed relative to the shaping head. For example, in the knee, osteophytes from posterior and other portions of the femoral condyle which are typically difficult to access can be easily contacted and shaped using apparatus 10 of the present invention.

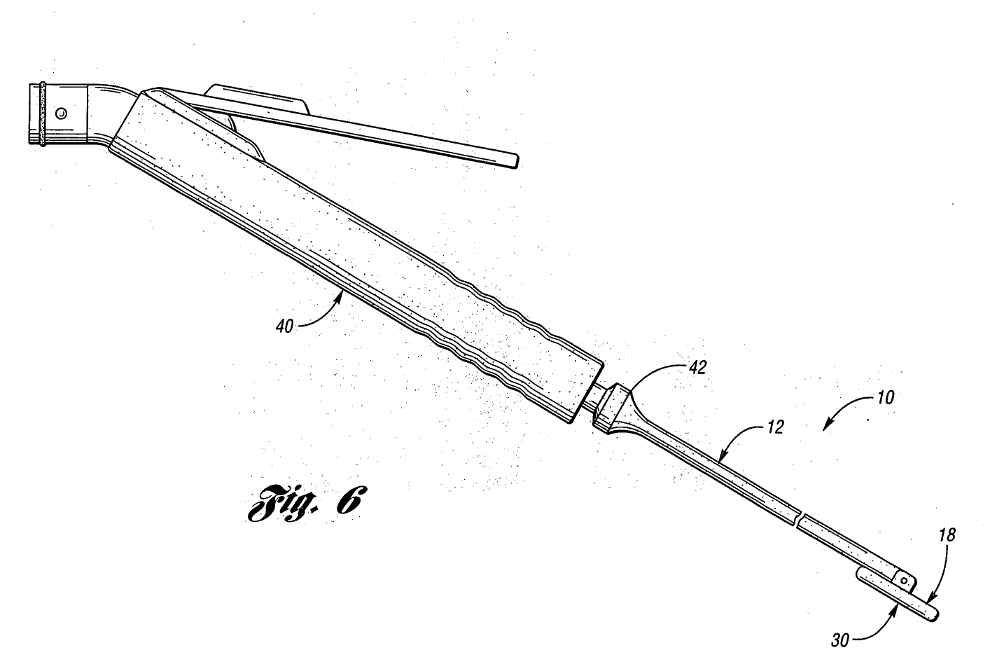

In addition to the hinge joint depicted in FIGS. 1-3, apparatus 10 according to the present invention could alternatively utilize a universal joint as depicted in FIG. 4. According to this aspect of the present invention, shaft distal end 16 is enlarged and hingedly connected to head 18 via two pins 24 offset by 90° to allow for pivotal movement between shaft 12 and head 18 along two different axes. Still further, it is contemplated that apparatus 10 of the present invention could include a ball joint between shaft 12 and head 18 to allow free movement in any direction, as shown in FIG. 5. Such a joint includes a ball member 26 on the shaft distal end 16 and a socket member 28 formed on the head 18 which is arranged to receive the ball member 26. Of course, ball member 26 could alternatively be provided on head 18 and socket member 28 on shaft distal end 16. In addition to the joints shown and described herein, it is understood that any type of joint which allows for pivotal movement of the head 18 relative to the shaft 12 is fully contemplated according to the present invention. Furthermore, while the various joints described and contemplated herein are shown to be at the distal end 16 of shaft 12 where shaft 12 and head 18 meet, it is understood that the joint can be at any location along shaft 12 for providing pivotal movement according to the present invention.

Shaping head 18 can have any shape appropriate for the intended purpose. A generally square or rectangular shape of head 18 as depicted herein are especially suited for use with a reciprocating or oscillating motion of shaft 12. For use in the knee joint, head 18 can have dimensions of between about 6 to 50 mm in length and between about 4 to 25 mm in width. Of course, head 18 can have any dimensions necessary for its desired use, and can have any thickness sufficient to provide desired rigidity. Other shapes for head 18 contemplated according to the present invention include, but are not limited to, spherical, round, cylindrical, hemispherical, ball-shaped, or hourglass, wherein these shapes are especially suited for use with a rotating motion of shaft 12.

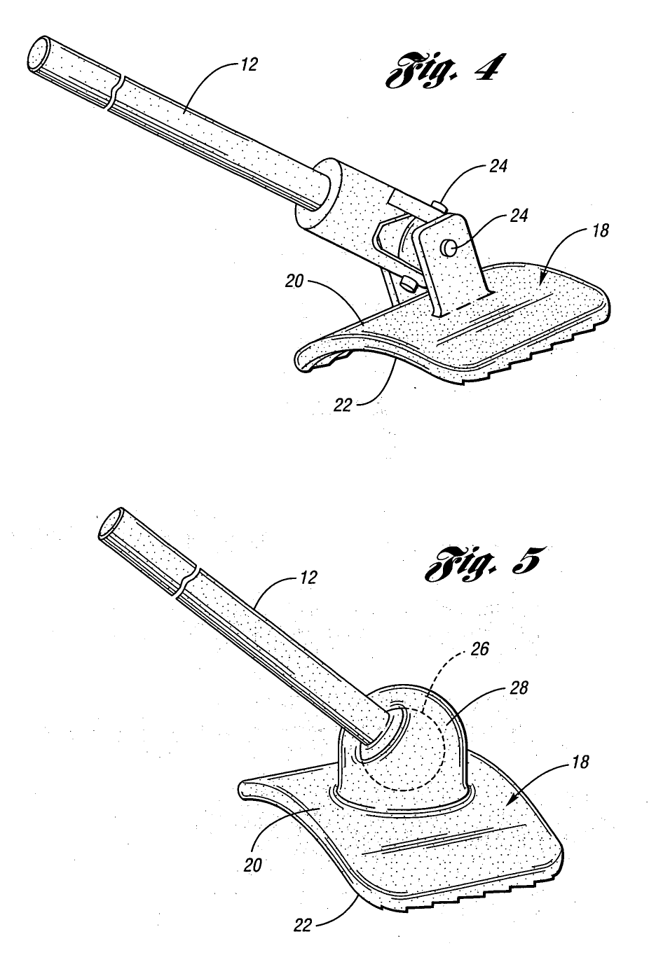

As best shown in FIGS. 2 and 3, head bottom surface 22 includes a textured portion 30 arranged to contact the joint surface desired to be shaped. Head top surface 20 could also include a textured portion 30 if desired. Textured portion 30 can have any configuration suitable for cutting and smoothing tissue, such as bone or cartilage, on the joint surface. With reference to FIG. 3, textured portion 30 can include cutting members such as ridges 32 to accomplish the shaping function, wherein ridges 32 can be formed as raised protrusions or etched into shaping head 18. Ridges 32 depicted herein are between about 0.1 to 2.0 mm thick and have a spacing of between about 0.2 to 5.0 mm. Ridges 32 can be configured so as to provide a relatively fine smoothing effect, or configured to provide a relatively coarser smoothing effect. For example, relatively shallow ridges 32 with larger spacing could be used for cutting and shaping cartilage, whereas relatively deeper ridges 32 with closer spacing could be used for cutting and shaping bone.

In addition to ridges 32, other types of cutting members, such as pegs, spikes, knurls, facets, or any other suitable members for shaping, cutting, or smoothing tissue within the joint cavity could alternatively be utilized. Textured portion 30 could also employ cutting members which include openings to allow debris to exit away from the joint surface, thereby providing a self-cleaning mechanism for apparatus 10. Textured portion 30 can be integrally formed with head 18 itself through machining, etching, stamping, or the like, or alternatively textured portion 30 can be a separate portion attached to head 18.

As depicted herein (see FIG. 1), head 18 can be generally concave and can include a medial-lateral radius 34, an anterior-posterior radius 36, or both. Advantageously, then, configurations of head 18 which utilize compound curves are possible with apparatus 10 according to the present invention, as opposed to single radius designs often found in the prior art. The configuration of head 18 can be selected according to the desired shape of the joint surface, and apparatus 10 can be utilized in a free-hand manner for shaping the joint surface without any need for fixation of the apparatus 10. For use in shaping the femoral condylar surface of the knee joint, medial-lateral radius 34 and/or the anterior-posterior radius 36 can be between about 5 to 60 mm. Alternatively, head 18 could be generally flat, or could be generally convex. Furthermore, the radius of curvature can be variable over head 18.

Apparatus 10 of the present invention can be constructed from any suitable material or combination of materials. For example, possible material choices include metals, such as stainless steel, polymeric materials, and any other suitably strong and durable materials.

With reference to FIG. 6, apparatus 10 according to the present invention is preferably also arranged to be releasably connected to a powered surgical instrument 40, such as a reciprocating, oscillating, rotating, or vibrating saw instrument as is known in the art (for example, reciprocating saws may include Hall Surgical Micro 110 and Stryker TPS System). As such, shaft 12 is sized appropriate to the instrument 40, typically about ⅛ inch in diameter. Also, depending on the manufacturer of the instrument 40, shaft 12 may also include notches or other locating means (not shown) to prevent unwanted shaft rotation or discharge from the instrument 40 during use. Shaft 12 can be secured to the powered surgical instrument 40 by inserting shaft 12 therein and tightening a collet 42 on the instrument 40 as is known in the art. Apparatus 10 according to the present invention can also be used with a separate support device or guide to control placement of the apparatus 10, such as in a manner similar to guides used with acetabular reamers as is known in the art.

The present invention further provides a method of shaping a mammalian joint surface comprising providing apparatus 10 as described above, and using the apparatus 10 to shape the joint surface, such as prior to providing a prosthesis within the joint cavity. With reference to the knee joint, for example, access to the joint cavity is obtained either arthroscopically or via arthrotomy, which provides an opening for the insertion of apparatus 10 to smooth the femoral and/or tibial surfaces, as well as provides access for a prosthesis to be inserted subsequently. Apparatus can be easily manipulated around and within the joint space due to the pivotal connection between shaft 12 and head 18, allowing cutting and smoothing of the joint surfaces. As described above, a guide can be used to aid in placement of apparatus 10. Following shaping, the joint can be thoroughly irrigated to remove any tissue debris.

While aspects of the invention have been illustrated and described, it is not intended that these aspects illustrate and describe all possible forms of the invention. Rather, the words used in the specification are words of description rather than limitation, and it is understood that various changes may be made without departing from the spirit and scope of the invention.

What is claimed is:

1. An apparatus for shaping a mammalian joint surface, comprising:
a shaft having a proximal end and a distal end;
a head pivotally connected to the shaft distal end, the head having a textured portion arranged to contact and shape the joint surface.

2. The apparatus according to claim 1, wherein the head is connected to the shaft distal end by a hinge joint.

3. The apparatus according to claim 1, wherein the head is connected to the shaft distal end by a universal joint.

4. The apparatus according to claim 1, wherein the head is connected to the shaft distal end by a ball joint.

5. The apparatus according to claim 1, wherein the head is generally rectangular.

6. The apparatus according to claim 1, wherein the head has dimensions of between about 6 to 50 mm in length and between about 4 to 25 mm in width.

7. The apparatus according to claim 1, wherein the head comprises one of a round, hemispherical, spherical, hourglass, cylindrical, or ball-shaped configuration.

8. The apparatus according to claim 1, wherein the head includes a medial-lateral radius of curvature between about 5 and 60 mm.

9. The apparatus according to claim 1, wherein the head includes an anterior-posterior radius of curvature between about 5 and 60 mm.

10. The apparatus according to claim 1, wherein the head includes a compound curvature.

11. The apparatus according to claim 1, wherein the head is generally concave.

12. The apparatus according to claim 1, wherein the head is generally flat.

13. The apparatus according to claim 1, wherein the joint surface includes at least one of bone and cartilage.

14. The apparatus according to claim 1, wherein the shaft is generally cylindrical.

15. The apparatus according to claim 1, wherein the textured portion includes a plurality of cutting members.

16. The apparatus according to claim 15, wherein the cutting members include openings for allowing tissue debris to exit away from the joint surface.

17. The apparatus according to claim 15, wherein the cutting members include ridges having a thickness of between about 0.1 to 2.0 mm and having a spacing of between about 0.2 to 5.0 mm.

18. The apparatus according to claim 1, wherein the textured portion is integrally formed with the head.

19. The apparatus according to claim 1, wherein the textured portion comprises a separate portion attached to the head.

20. The apparatus according to claim 1, wherein the apparatus is constructed at least partially from a metallic material.

21. The apparatus according to claim 1, wherein the apparatus is constructed at least partially from a polymeric material.

22. The apparatus according to claim 1, wherein the shaft is arranged to be connected to a powered surgical instrument.

23. The apparatus according to claim 1, wherein the apparatus is arranged for use in shaping at least one of a femoral surface and a tibial surface of the knee.

24. A rasp for shaping a surface within a knee joint, comprising:
an elongated shaft having a proximal end and a distal end; and
a generally concave shaping head pivotally connected to the shaft distal end, the head having cutting members for contacting and shaping the knee joint surface.

25. A method of shaping a mammalian joint surface, the method comprising:
providing an apparatus including a shaft having a proximal end and a distal end, and a head pivotally attached to the shaft distal end, the head having a textured portion; and
placing the textured portion of the apparatus in contact with the joint surface for shaping the joint surface.

26. The method according to claim 25, further comprising making an incision through which the apparatus is inserted into a joint cavity.

27. The method according to claim 25, further comprising shaping the joint surface without requiring fixation of the apparatus to any joint surface.

28. The method according to claim 25, wherein placing the textured portion in contact with the joint surface includes pivoting the shaft relative to the head.

29. The method according to claim 25, further comprising using a guide to aid in placement of the apparatus.

30. The method according to claim 25, further comprising connecting the shaft proximal end to a powered surgical instrument.

31. The method according to claim 25, wherein shaping the joint surface includes maneuvering the apparatus in a free-hand manner.

32. The method according to claim 25, wherein shaping the joint surface includes shaping at least one of bone or cartilage.

33. The method according to claim 25, wherein shaping the joint surface includes shaping at least one of a femoral surface and a tibial surface of the knee.

34. The method according to claim 25, further comprising implanting a prosthesis following shaping of the joint surface.
